(19) United States
(12) Patent Application Publication
(10) Pub. No.: US 2008/0077157 A1
(43) Pub. Date: Mar. 27, 2008

(54) INSERTION APPARATUS HAVING A CONCAVE SURFACE

(75) Inventor: Erik W. Peterson, Walnut Creek, CA (US)

Correspondence Address: MICHAEL BEST & FRIEDRICH LLP, 100 E WISCONSIN AVENUE, Suite 3300, MILWAUKEE, WI, 53202, US

(73) Assignee: MEDICAL INSTRUMENT DEVELOPMENT LABORATORIES, INC., San Leandro, CA (US)

(21) Appl. No.: 11/460,041

(22) Filed: Jul. 26, 2006

(51) Int. Cl. A61F 9/00

(52) U.S. Cl. 606/107

(57) ABSTRACT

An ophthalmic surgical apparatus for inserting a cannula into an incision. In one embodiment, the apparatus includes a needle extending in an axial direction. The needle has a manipulation portion disposed at a first end of the needle and an insertion portion disposed at a second end of the needle. The insertion portion includes a receiving side, and the receiving side has a concave surface and a tip. In one embodiment, the insertion portion has an s-curve shape.

### FIELD OF THE INVENTION

Embodiments of the invention relate to aspects of improved trocar-cannula devices for use in surgery of the posterior segment of the eye.

### BACKGROUND OF THE INVENTION

A trocar-cannula is a surgical instrument. It can be used to drain fluid from a body cavity, introduce fluids into the body cavity, and insert a tool into the body cavity to perform surgical functions. The trocar-cannula is comprised of two principal parts: (1) a hollow tube or cannula and (2) a puncturing member referred to as an obturator or trocar. The cannula is inserted through the wall of the body cavity with the assistance of the trocar as it is passed through the cannula.

A trocar-cannula may be used in cardiovascular surgery, laproscopic surgery, arthroscopic surgery, and intraocular surgery. However, the construction of the trocar-cannula is generally different for each application. For example, an arthroscopic trocar-cannula is usually too large for intraocular surgery. Furthermore, it may contain projections on the exterior surface of the lumen to prevent the cannula from inadvertently disengaging the joint. These projections could cause tissue damage if applied to the eye or other parts of the body.

In intraocular surgery, a trocar-cannula is often used to obtain access to the posterior-segment of the eye (the area behind the lens). Although surgical instruments can be introduced directly through incisions in the sclera, some surgeons introduce instruments through a cannula to reduce damage to the incision and nearby tissues.

In intraocular surgery, an alternative method is sometimes used to place a cannula into the eye. An incision through the sclera is first formed by means of a surgical knife, and the cannula is then introduced into the incision by means of an insertion instrument similar to a trocar, but with a blunt tip. The incision made with the knife is less traumatic and heals more rapidly than the puncture made with the three-edged trocar.

### SUMMARY OF THE INVENTION

One disadvantage of this alternative method is that the knife incision can be difficult to locate after the knife is removed. Thus, one object of certain embodiments of the invention is to provide an insertion instrument which can be guided into the incision before the knife is removed.

In one particular embodiment, the invention is directed to an ophthalmic surgical device for inserting a cannula. The device includes a needle extending in an axial direction. The needle has a manipulation portion and an insertion portion disposed at one end of the needle. The insertion portion includes a receiving side, and the receiving side has a concave surface and a tip.

Another aspect of the invention is a method of inserting an instrument into a scleral incision. The method includes making a scleral incision with a cutting implement, receiving a convex surface of the cutting implement onto a concave surface of an insertion apparatus, inserting the tip of the insertion apparatus into the scleral incision by movement along the cutting implement, removing the cutting implement from the scleral incision, advancing the insertion apparatus into the scleral incision, and removing the insertion apparatus from the scleral incision.

Another aspect of the invention is a method for placing a device into an opening. The method includes the steps of creating an opening with a cutting implement, receiving a convex surface of the cutting implement onto a concave surface of an insertion apparatus for placing the device into the opening, inserting a needle of the insertion apparatus into the opening, removing the cutting implement from the opening, placing the device into the opening, and removing the insertion apparatus from the opening.

Another embodiment of the invention provides an apparatus for placing a device in an opening. The apparatus includes a hollow needle having a first end and a second end. The needle has a manipulation portion at the first end of the needle and an s-curve shaped insertion portion at the second end of the needle. The s-curve shaped insertion portion includes a concave receiving surface and a tip.

Another embodiment of the invention provides a surgical kit. The surgical kit includes a knife and one or more cannula-insertion instruments. The knife includes a convex portion and a cutting edge. The cannula-insertion instrument includes a hollow needle that has first and second ends, a manipulation portion at the first end of the needle and an insertion portion at the second end of the needle. The insertion portion has a concave surface sized and shaped to receive the convex portion of the knife and a tip. A cannula is disposed co-axially over the hollow needle.

These and other aspects of various embodiments of the invention, together with the organization and operation thereof, will become apparent from the following detailed description when taken in conjunction with the accompanying drawings.

### BRIEF DESCRIPTION OF THE DRAWINGS

FIG. 1 is a perspective view of an exemplary insertion device or trocar embodying aspects of the invention.

FIG. 2A is a side view of the needle of the device of FIG. 1.

FIG. 2B is a top view of the needle of the device of FIG. 1.

FIG. 3 is a perspective view of a knife that may be used in conjunction with the insertion device of FIG. 1.

FIG. 4 is a perspective view of the insertion device of FIG. 1 and the knife of FIG. 3 used in a surgical procedure performed on an eye.

FIG. 5 is another perspective view of the insertion device of FIG. 1 and the knife of FIG. 3 shown in a second position when used in a surgical procedure on an eye.

FIG. 5A is a detailed view of the mating of the concave surface of the insertion device of FIG. 1 with the convex surface of the knife of FIG. 3.

FIG. 6 is a perspective view of the insertion device of FIG. 1 as used to place a cannula in an incision made in an eye.

FIG. 6A is a perspective view of the eye following insertion of the cannula.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of illustration and description of one or more examples of the invention and should not be regarded as limiting. It is possible that the invention could be embodied in forms not specifically described herein.

### DETAILED DESCRIPTION

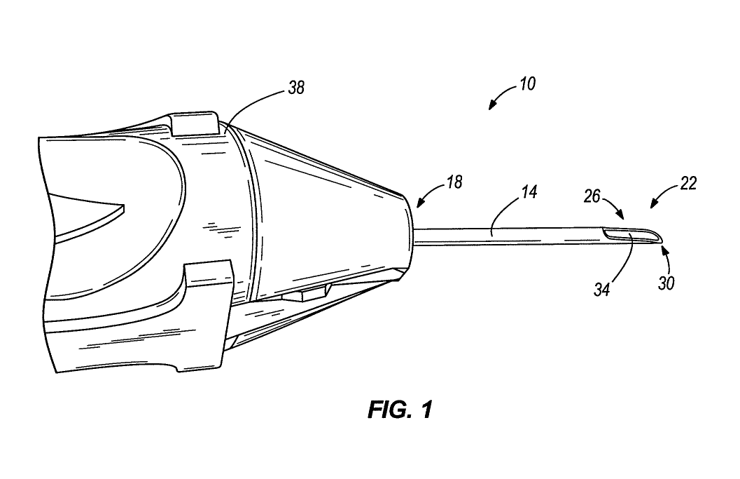

With reference to FIG. 1, there is shown an insertion apparatus 10 for use with an ophthalmic cannula. The insertion apparatus 10 comprises a needle 14. The needle 14 is a hollow tube with a manipulation portion 18 and an insertion portion 22 disposed at opposite ends of the needle 14. The insertion portion 22 further includes a receiving side 26 and a tip 30. The receiving side 26 has a concave surface 34. The receiving side 26 is manufactured with a polished, smooth, or machine-finished surface. The receiving side 26 is configured to slidingly receive another instrument, such as a cutting implement described below. In some embodiments, a handle 38 may be coupled to the manipulation portion 18 to facilitate easier maneuvering and manipulation of the apparatus 10 by a user.

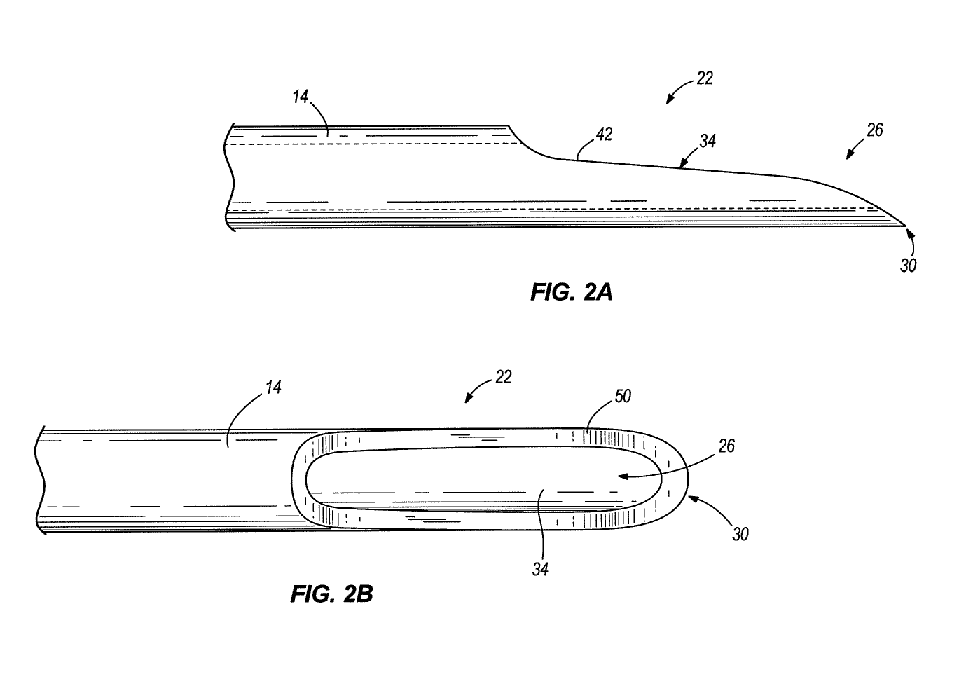

In some embodiments and as illustrated in FIG. 2A, the insertion portion 22 has an s-curve shape 42 when viewed from the side. The s-curve shape 42 can be machined into the needle 14. In particular, when viewed from the side, the needle sidewalls 50 (shown in FIG. 2B) resemble an s-curve shape 42. An s-curve shape 42 generally has a two c-shape curves, each c-shape curving in a direction opposite to the other curve. In other embodiments, the insertion portion 22 can have other shapes corresponding to the concave surface 34 of the insertion portion 22 and which complements other instruments that may be used in conjunction with the insertion apparatus 10. In some embodiments, the insertion portion 22 has a wedge-shaped tip 30. In other embodiments, the tip 30 can be other shapes that facilitate insertion into an opening, such as an opening in the sclera of an eye 66.

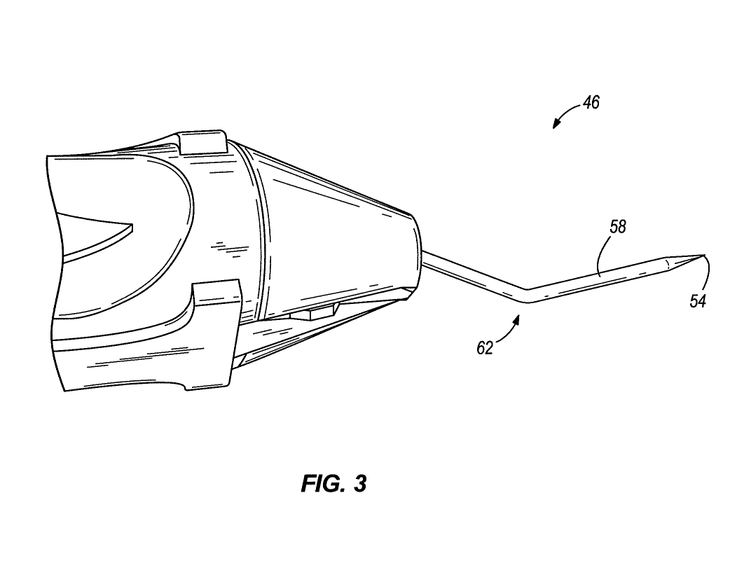

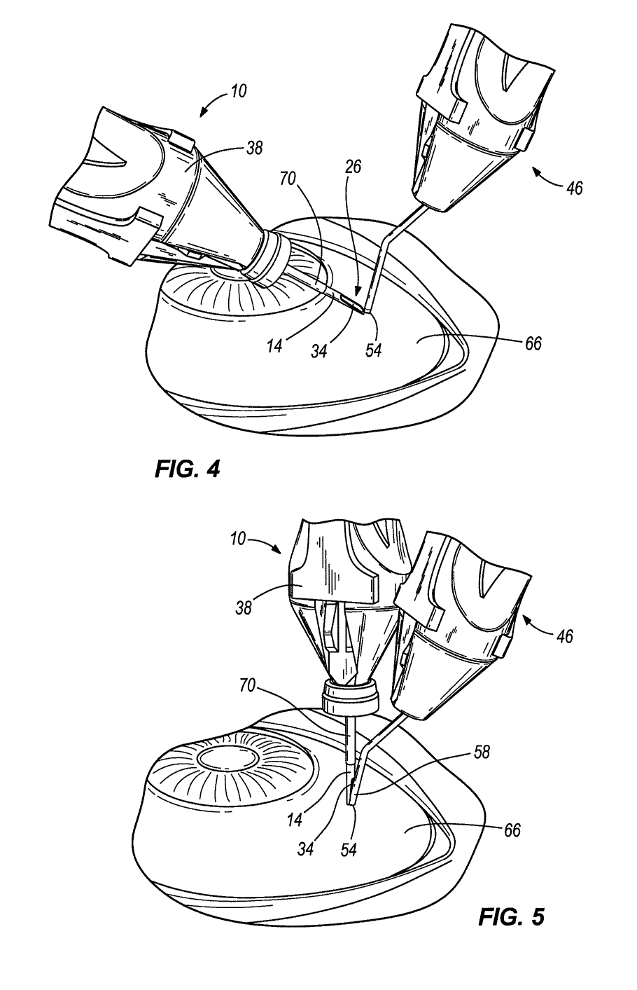

In the embodiments illustrated in FIGS. 4-6A, the insertion apparatus 10 includes features that facilitate its use in conjunction with another instrument. In some embodiments and as illustrated in FIG. 4, a cutting implement or knife 46 is used to make an opening. As illustrated in FIG. 3, the knife 46 has a blade 54 and a convex surface 58. The blade 54 of the knife 46 has a double bend 62 to facilitate using one or more instruments simultaneously within the same opening.

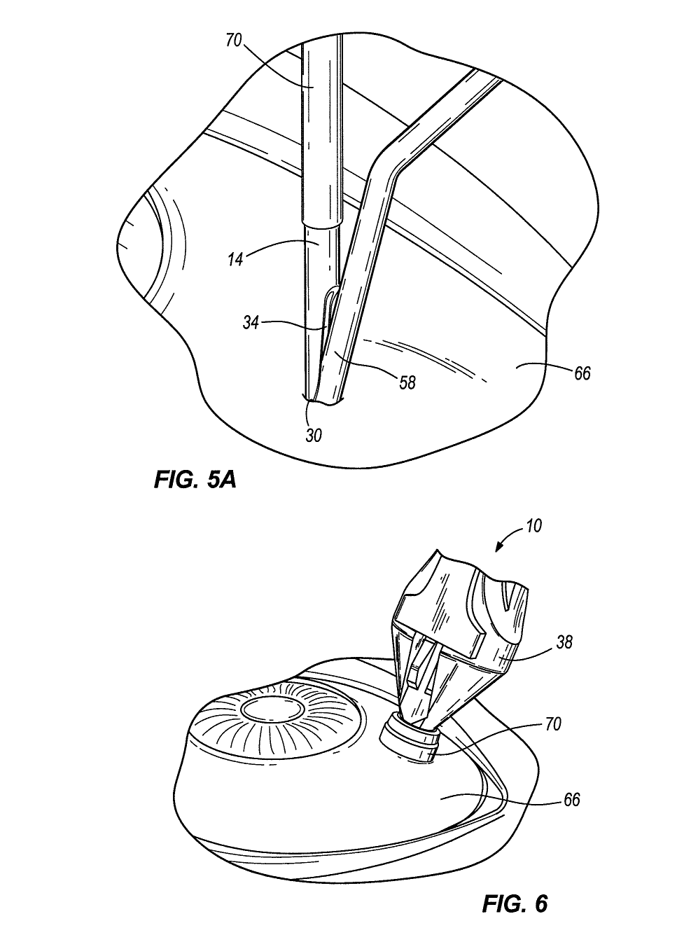

As illustrated in FIG. 4, the knife 46 is used to make an incision through both the conjunctival and scleral membranes of an eye 66. In one method of using the insertion apparatus 10 and the knife 46, after the blade 54 of the knife 46 penetrates the conjunctiva and the sclera, the knife 46 is partially withdrawn from the incision so that the blade 54 remains in the scleral incision. At this point and as illustrated in FIG. 5, the tip of the insertion apparatus 10 is inserted into the conjunctival and scleral incisions.

As illustrated in FIG. 5, a cannula 70 or similar device can be positioned on the needle 14. The diameter of the central opening of the cannula 70 is slightly larger than that of the needle 14 so that the cannula 70 may be slid over the needle 14 and held in place on the needle 14 by frictional forces. The cannula 70 is further positioned on the needle 14 to be slid into the scleral opening upon placement of the insertion apparatus 10 into the opening. In some embodiments, the needle is sized or dimensioned such that when the cannula 70 is positioned on the needle 14, the needle 14 extends approximately 3.5 millimeters beyond the end of the cannula 70. Dimensioning the needle 14 in this manner is useful because the distance from the edge of the cornea to the optimal location of the incision on the average eye is approximately 3.5 millimeters. Thus, when so dimensioned, the needle 14 can be used as a guide to insure proper location of an incision. In other embodiments, the needle 14 can extend more or less than 3.5 millimeters beyond the end of the cannula 70.

illustrated in FIGS. 5 and 5A, as the insertion apparatus 10 is positioned for insertion into the conjunctival and scleral incisions, the concave surface 34 of the receiving side 26 of the insertion portion 22 is slidingly received on the convex surface 58 of the knife 46. The concave surface 34 of the receiving side 26 of the insertion portion 22 is configured to slidingly receive the convex surface 58 of the knife 46. The mating of the concave surface 34 of the insertion portion 22 with the convex surface 58 of the knife 46 facilitates the guiding of the insertion apparatus 10 into the opening.

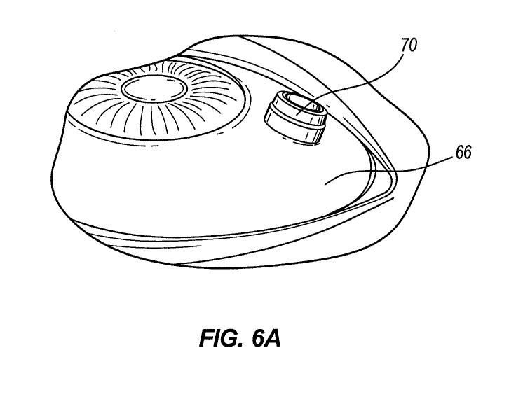

Once the tip 30 of the needle 14 is moved into the scleral incision, the knife 46 is gradually removed from the scleral incision. As illustrated in FIG. 6, the handle 38 of the insertion apparatus 10 is then pressed forward to further drive the needle 14 and the cannula 70 into the eye 66. As illustrated in FIG. 6A, once the cannula 70 is properly in place, the insertion apparatus 10 is removed from the eye 66.

Various features of the invention are set forth in the following claims.

What is claimed is:

1. An ophthalmic surgical apparatus for inserting a cannula into an incision, the apparatus comprising:
a needle extending in an axial direction, the needle having
a manipulation portion disposed at a first end of the needle; and
an insertion portion disposed at a second end of the needle, the insertion portion including:
a receiving side, the receiving side having a concave surface; and
a tip.

2. The ophthalmic surgical apparatus of claim 1, wherein the insertion portion has an s-curve shape.

3. The ophthalmic surgical apparatus of claim 1, wherein the receiving side is configured to slidingly receive a surgical implement.

4. The ophthalmic surgical apparatus of claim 1, wherein the tip is wedge-shaped.

5. The ophthalmic surgical apparatus of claim 1, further comprising a cannula positioned on the needle.

6. The ophthalmic surgical apparatus of claim 5, wherein the needle is dimensioned to extend at least 3.5 millimeters beyond the cannula.

7. The ophthalmic surgical apparatus of claim 1, further comprising a handle cooperating with the manipulation portion.

8. A method of inserting an instrument into a scleral incision, the method comprising:
making a scleral incision with a cutting implement;
receiving a convex surface of the cutting implement onto a concave surface of an insertion apparatus;
inserting a tip of the insertion apparatus into the scleral incision by movement along the cutting implement;
removing the cutting implement from the scleral incision;
advancing the insertion apparatus fully into the scleral incision; and
removing the insertion apparatus from the scleral incision.

9. The method of claim 8, further comprising the step of placing a cannula over a needle of the insertion apparatus.

10. The method of claim 9, further comprising the step of forming the needle so that the needle extends at least 3.5 millimeters beyond an end of the cannula.

11. The method of claim 8, further comprising the step of forming the concave surface of the insertion apparatus to have an s-curve shape.

12. The method of claim 8, further comprising the step of forming the insertion apparatus to have a wedge-shaped tip.

13. The method of claim 12, further comprising sharpening the wedge-shaped tip.

14. The method of claim 8, further comprising mating the concave surface of the insertion apparatus with the convex surface of the cutting implement to guide the insertion apparatus into the scleral incision.

15. A method for placing a device into an opening, the method comprising the steps of:
creating an opening with a cutting implement;
receiving a convex surface of the cutting implement onto a concave surface of an insertion apparatus for placing the device into the opening;
inserting a needle of the insertion apparatus into the opening;
removing the cutting implement from the opening;
placing the device into the opening; and
removing the insertion apparatus from the opening.

16. The method of claim 15, further comprising placing the device over the needle of the insertion apparatus.

17. The method of claim 16, further comprising the step of forming the needle of the insertion apparatus so that the needle extends at least 3.5 millimeters beyond an end of the device.

18. The method of claim 15, further comprising the step of forming an s-curve shape on the concave surface of the insertion apparatus.

19. The method of claim 15, further comprising the step of mating the concave surface of the insertion apparatus with the convex surface of the cutting implement to guide the insertion apparatus into the opening.

20. An apparatus for placing a device in an opening, the apparatus comprising:
a hollow needle having a first end and a second end, the needle having
a manipulation portion at the first end of the needle; and
an s-curve shaped insertion portion at the second end of the needle, the s-curve shaped insertion portion comprising:
a concave receiving surface; and
a tip.

21. The apparatus of claim 20, further comprising a cannula positioned on the needle.

22. The apparatus of claim 21, wherein the needle extends at least 3.5 millimeters beyond an end of the cannula.

23. The apparatus of claim 20, wherein the concave receiving side is configured to receive a convex side of an other apparatus.

24. A surgical kit comprising
a knife having a convex portion and a cutting edge; and
an insertion apparatus, the insertion apparatus including
a hollow needle having first and second ends, a manipulation portion at the first end of the needle and an insertion portion at the second end of the needle,
the insertion portion having a concave surface sized and shaped to receive the convex portion of the knife and a tip.

25. The surgical kit of claim 24, wherein the insertion portion has an s-curve shape.

26. The surgical kit of claim 24, further comprising a cannula positioned on the needle.

27. The surgical kit of claim 26, wherein the needle is dimensioned to extend at least 3.5 millimeters beyond the cannula.

28. The surgical kit of claim 24, further comprising a handle cooperating with the manipulation portion.
